(19) United States
(12) Patent Application Publication
(10) Pub. No.: US 2023/0086800 A1
(43) Pub. Date: Mar. 23, 2023

(54) ANTIBODY FRAGMENTS CONJUGATED TO PEG-PLGA NANOPARTICLES IMPROVE IMMUNOTHERAPY AGAINST CANCER CELLS

(71) Applicant: The Board of Trustees of the Leland Stanford Junior University, Stanford, CA (US)

(72) Inventors: Sibu Kuruvilla, Redwood City, CA (US); Dean W. Felsher, San Mateo, CA (US); Christina Kim Lee, Redwood City, CA (US)

(21) Appl. No.: 17/904,562

(22) PCT Filed: Mar. 8, 2021
(86) PCT No.: PCT/US2021/021329

§ 371 Date: Aug. 18, 2022

Related U.S. Application Data
(60) Provisional application No. 62/986,077, filed on Mar. 6, 2020.

(51) Int. Cl. A61K 47/69; A61K 47/59; A61K 47/60; A61K 47/68; A61P 35/00; C07K 16/28

(52) U.S. Cl. CPC A61K 47/6937; A61K 47/593; A61K 47/60; A61K 47/6849; A61P 35/00; C07K 16/2827; A61K 2039/505

(57) ABSTRACT

Anti-PD-1/PD-L1 antibody conjugated nanoparticles and methods of treating cancer, including without limitation hepatocellular carcinoma, are provided. The conjugates comprise antibodies, e.g. antibody F(ab) fragments, covalently linked to nanopartides. The antibody conjugated nanoparticles provide high tumor-specific delivery by extending circulation time of the antibodies by increasing their geometry and removing the Fc portion, and minimizing off-target distribution and toxicity. In some embodiments the antibody conjugated nanoparticlesprovide for increased therapeutic efficacy, e.g. in decreased tumor growth, relative to unconjugated antibody, or relative to unconjugated F(ab) fragments of an antibody.

### CROSS REFERENCE TO RELATED APPLICATION

The present application claims the benefit of and priority to U.S. Provisional Patent Application No. 62/986,077, filed Mar. 6, 2020, the entire disclosure of which is hereby incorporated by reference herein in its entireties for all purposes.

### BACKGROUND

Immunotherapy through blockade of the PD-1/PD-L1 axis has recently exhibited success in the clinic leading to FDA approval in many tumor indications, but considerable efficacy and tolerability against solid tumors has yet to be observed. For example, hepatocellular carcinoma (HCC), the most common form of primary liver cancer, is the 3rd leading cause for cancer-related deaths worldwide but has yet to experience substantial success from immunotherapy. Nivolumab and pembrolizumab, checkpoint inhibitors against PD-1, were approved in 2018 as 2nd-line therapies for Sb-refractory HCC patients. Despite promising results, nivolumab unfortunately suffers from poor patient response rates (<18%) and treatment-related adverse events in 70-80% of patients (23-29% grade 3/4). Pembrolizumab was investigated as a monotherapy in a Phase II trial (KEYNOTE-224, NCT02702414) as a 2nd-line therapy for previously-treated advanced HCC patients and achieved an objective response rate (ORR) of 17% (REF), but a follow-up Phase III trial (KEYNOTE-240, NCT02702401) failed to meet the primary endpoints (REF) in the same subset of patients. Additionally, other checkpoint inhibitors like PD-L1 and CTLA-4 have seen minimal efficacy against HCC.

The hurdles monoclonal antibodies (mAbs) face in HCC therapy can be attributed to poor tumor-specific distribution, rapid clearance (half-life of 24 hours to 3 days), and off-target distribution/toxicity (grade ¾ adverse events in 23-55% of patients). It has been reported that approximately 10% of the injected dose of mAb reached solid tumor tissue. Further, due to the geometric properties of mAbs (5.2-7.1 nm), they are rapidly cleared through glomerular filtration in the kidneys (<10 nm). The recognition of the Fc portion of the mAb by the Fc-receptor on several immune cell types (e.g. macrophages, dendritic cells, natural killer cells, and B-cells) leads to rapid recognition and removal by these cells to their respective immune compartments. This clearance then contributes to the off-target immune-related toxicity observed in patients (e.g. colitis, hepatitis, pneumonitis, etc.). Therefore, it is evident that novel formulations and unique strategies for checkpoint inhibition is necessary to achieve their full therapeutic potential against HCC.

### SUMMARY OF THE INVENTION

Antibody conjugated nanoparticles and methods of treating cancer, including without limitation hepatocellular carcinoma, are provided. The conjugates comprise antibodies covalently linked to nanoparticles, usually an antigen binding antibody fragment lacking an Fc region, e.g. an F(ab) or F(ab)′, F(ab)2 fragment, etc. The antibody conjugated nanoparticles provide high tumor-specific delivery by extending circulation time of the antibodies by increasing their geometry and removing the Fc portion, and minimizing off-target distribution and toxicity. In some embodiments the antibody conjugated nanoparticlesprovide for increased therapeutic efficacy, e.g. in decreased tumor growth, extended time of effectiveness, for example to increase activity of desirable immune cell populations after administration, etc., relative to unconjugated antibody, or relative to unconjugated F(ab) fragments of an antibody.

In some embodiments an antibody specific for PD-L1 or PD-1 is loaded on nanoparticles for drug delivery. In some embodiments the nanoparticles are PLGA particles coated with PEG. In some embodiments the antibody conjugated nanoparticles are formulated in a physiologically acceptable excipient. In some embodiments the antibody is a human, humanized or chimeric antibody specific for human PD-L1. In some embodiments the antibody is selected from Atezolizumab; Avelumab; and Durvalumab. In some embodiments the antibody conjugate is provided in a unit dose formulation.

In some embodiments the antibody conjugated nanoparticles described herein and administered to a patient for treatment of cancer. In some embodiments the antibody conjugated nanoparticle is delivered intra-peritoneally. In some embodiments the cancer is a solid cancer. In some embodiments the cancer is a carcinoma. In some embodiments the carcinoma is a hepatocellular carcinoma. In some embodiments the hepatocellular carcinoma is myc-driven. In some embodiments the hepatocellular carcinoma is assessed by myc dependence prior to selection for treatment with an antibody conjugated nanoparticle as disclosed herein. The methods of the invention can provide for increased overall survival of the individual being treated.

In some embodiments the antibody conjugated nanoparticles are provided as a single agent therapy. In some embodiments the nanoparticles are combined with additional anti-cancer agents, including without limitation: immunotherapy, radiation therapy, chemotherapy, surgery, etc.

In some embodiments, the antibody conjugated to the nanoparticle is an F(ab) fragment. In some embodiments the F(ab) fragment is covalently bound to the nanoparticle. In some embodiments the F(ab) fragment is covalently bound to PEG in a nanoparticle comprising PLGA-PEG. In some embodiments the F(ab) fragment is covalently bound through a free thiol group to a maleimide linker joined to PEG in a nanoparticle comprising PLGA-PEG. The F(ab) fragment may be synthesized as such, or may be a digest on the original, intact antibody.

In some embodiments the antibody conjugate is prepared by conjugated F(ab) fragments comprising a free thiol with MAL-PEG-PLGA monomers. The PEG may be of a molecular weight from about 1k to about 20k, e.g. 1k, 2k, 5k, 10, 15k, 20k. The PLGA may be of a molecular weight from about 1k to about 20k, e.g. 1k, 2k, 5k, 10, 15k, 20k. In some embodiments the monomer is MAL-PEG(5k)-PLGA(5k). In some embodiments the antibody fragment is combined with the monomers at a ratio of from about 10:1, 5:1, 2:1, 1:1; 1:2; 1:5; 1:10; 1:20 F(ab) to monomer ratio. The F(ab) conjugated monomer forms nanoparticles by an oil and water emulsion method. The resulting nanoparticles may have an average diameter of from about 100-500 nM, from about 200-300 nM, and may be around 240-270 nM. The potential of the antibody conjugate nanoparticle may be from about −20 to about −30 mV.

### BRIEF DESCRIPTION OF THE DRAWINGS

FIG. 1. Synthesis of PD-L1-PEG-PLGA NPs

FIG. 2. Biodistribution of PD-L1-PEG-PLGA NPs

FIG. 3. Therapeutic Efficacy of PD-L1-PEG-PLGA NPs in MC38 Tumor Model

FIG. 4. MALDI-TOF of PD-L1 Fragmentation.

FIG. 5. Stability of nanoparticle over time. Antibody carrying nanoparticle size decreased to a size comparable to that of empty nanoparticle by week 3.

FIGS. 6A-6B. Antibody carrying nanoparticles injected IP significantly reduced tumor growth compared to antibody carrying nanoparticles injected through IV.

FIGS. 7A-7B. Immune landscape change over time. Elevated CD4⁺T cells and B cells were found in the spleen of mice that received antibody carrying nanoparticle compared to the spleen of mice that received intact nanoparticle at week 3 post MC38 injection.

FIG. 8. Nanoparticle toxicity. Treatment with antibody carrying nanoparticle did not show notable toxicity to tumor bearing mice compared to the intact PD-L1 treatment.

### DETAILED DESCRIPTION OF THE EMBODIMENTS

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Nanoparticles (NPs) are effective in overcoming delivery issues faced by a variety of therapeutic payloads. Due to their unique physicochemical properties and larger size over small molecules, NPs can protect therapeutic payloads and improve their biodistribution properties by eliminating rapid renal clearance. The ability to coat the NP surface with water-soluble, “stealth-like” polymers such as poly(ethylene glycol) (PEG) further protects the payload from rapid clearance and enhances distribution properties. NPs can also enable tumor-specific delivery of the payload through both passive and active targeting to tumor tissue. Passive targeting is allowed by the enhanced permeation and retention (EPR) effect due the leaky vasculature and the compromised lymphatic system of tumor tissue. Active targeting can be achieved by the display of tumor-specific targeting ligands or antibodies. Finally, NPs have high functionality which allows co-loading of multiple therapeutic molecules, such as chemotherapeutic drugs, genetic material, and imaging modalities. PEG-PLGA (poly(ethylene glycol)-poly(lactic-co-glycolic acid)) NPs are particular interest.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases, or provided as an intact tetramer comprising variable regions, hinge and Fc region sequences. Thus pepsin digests an antibody below the disulfide linkages in the hinge region to produce F(ab)′₂, a dimer of Fab which itself is a light chain joined to VH-CH, by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin, e.g. IgG1, IgG2a, IgG2b, IgG3, IgG4, etc.

Antibodies of interest for the present disclosure include antibodies specific for the immune-checkpoint proteins PD1 and PDL1. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand. Because many tumors are highly infiltrated with T_{Reg} cells, blockade of the PD1 pathway may also enhance antitumor immune responses by diminishing the number and/or suppressive activity of intratumoral T PD-1 inhibitors.

In some embodies, an antibody comprises an human, humanized or chimeric antibody specific for human PD-1 or human PDL-1. In some embodiments the antiboy is a clinically approved antibody.

Antibodies specific for PD-1 include: Pembrolizumab (Keytruda™); Nivolumab (Opdivo™); spartalizumab (PDR001); and Cemiplimab (Libtayo™); Camrelizumab; and Toripalimab. Approved dosing for these antibodies include dose/kg weight, or flat rate dosing, and include intravenous and subcutaneous delivery. Currently approved dosage regimens can be accessed at, for example, FDA guidelines. Included are doses from about 0.5 to about 20 mg/kg; e.g. 1 mg/kg; 2 mg/kg, 3 mg/kg, 5 mg/kg; 10 mg/kg; 20 mg/kg; administered every two weeks, every three weeks, monthly, etc. Flate rate doses currently approved include from about 60 mg to about 300 mg, e.g. 60 mg., 200 mg, 240 mg., etc., administered every two weeks, every three weeks, every 4 weeks, etc.

Antibodies specific for PD-L1 include: Atezolizumab (Tecentriq); Avelumab (Bavencio); Durvalumab (lmfinzi). Currently approved dosage regimens can be accessed at, for example, FDA guidelines. Included are doses from about 1 to about 25 mg/kg; administered every two weeks, every three weeks, monthly, etc. Flate rate doses may be from about 500 mg to about 2000 mg, from about 800 to about 1680 mg, e.g. 800 mg., 840 mg, 1200 mg., 1680 mg. etc., administered every two weeks, every three weeks, every 4 weeks, etc.

Examples of clinical trials and uses include, for example, Nivolumab: Deficiency mismatch repair (dMMR) or MSI-H metastatic colorectal cancer, NCT02060188; Melanoma, NCT01721746; Metastatic squamous Non-small-cell lung carcinoma (NSCLC), NCT01673867; Metastatic non-squamous NSCLC, NCT01673867; Locally advanced or metastatic urothelial carcinoma (UC), NCT02387996; Advanced Renal cell carcinoma, NCT01668784; Hematologic malignancy, NCT01592370, NCT02181738; Advanced hepatocellular Carcinoma, NCT01658878; Recurrent/Metastatic Head and neck squamous cell carcinoma (HNSCC), NCT02105636. Pembrolizumab: Advanced or unresectable melanoma, NCT01295827; Advanced or metastatic PD-L1-positive NSCLC, NCT01295827; Locally advanced or metastatic UC, NCT02335424, NCT02256436; Recurrent or metastatic HNSCC, NCT01848834; Hematologic malignancy, NCT02181738; Microsatellite instability or mismatch repair deficient cancers, NCT01876511; Advanced gastroesophageal Cancer, NCT02335411; Metastatic Cervical Cancer, NCT02628067; Locally advanced or metastatic, esophagus squamous cell carcinoma (ESCC), NCT02559687, NCT02564263. Cemiplimab: Advanced cutaneous squamous cell carcinoma (CSCC), NCT02383212, NCT02760498. Camrelizumab: Classical Hodgkin lymphoma (cHL), NCT03155425. Toripalimab: Malignant melanoma, NCT03013101. Avelumab: Locally advanced or metastatic UC, NCT01772004; Metastatic Merkel cell carcinoma, NCT02155647. Atezolizumab: Previously treated metastatic NSCLC, NCT01903993, NCT02008227; Locally advanced and metastatic UC; NCT02108652. Durvalumab: Locally advanced, unresectable NSCLC, NCT02125461; Locally advanced or metastatic UC, NCT01693562. In some embodiments the antibody conjugates of the invention provide for an effective dose substantially similar to the FDA approved dosing. In some embodiments the targeting and efficacy of the antibody conjugates allows for dosing at a lower dose, e.g. from about 0.1 to about 1 mg/kg.

Immune Responsiveness Modulators. Immune checkpoint proteins are immune inhibitory molecules that act to decrease immune responsiveness toward a target cell, particularly against a tumor cell in the methods of the invention. Endogenous responses to tumors by T cells can be dysregulated by tumor cells activating immune checkpoints (immune inhibitory proteins) and inhibiting co-stimulatory receptors (immune activating proteins). The class of therapeutic agents referred to in the art as “immune checkpoint inhibitors” reverses the inhibition of immune responses through administering antagonists of inhibitory signals. Other immunotherapies administer agonists of immune costimulatory molecules to increase responsiveness. In some embodiments, an in vitro assay of T cell activation, including without limitation assays in the Examples, is used in the determination of specific combinations and dosing schedules.

The two ligands for PD1 are PD1 ligand 1 (PDL1; also known as B7-H1 and CD274) and PDL2 (also known as B7-DC and CD273). The PD1 ligands are commonly upregulated on the tumor cell surface from many different human tumors. On cells from solid tumors, the major PD1 ligand that is expressed is PDL1. PDL1 is expressed on cancer cells and through binding to it's receptor PD1 on T cells it inhibits T cell activation/function. Therefore, PD1 and PDL1 blocking agents can overcome this inhibitory signaling and maintain or restore anti-tumor T cell function.

PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell function. However, since PDL1 is expressed on tumor cells, antibodies that bind and block PDL1 can also enable ADCP, ADCC, and CDC of tumor cells.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

In some embodiments, the cancer is hepatocellular carcinoma. Hepatocellular carcinoma is the most common type of primary liver cancer, with an estimated 23,000 new cases and about 14,000 deaths expected in 2012 in the US. However, it is more common outside the US, particularly in East Asia and sub-Saharan Africa where the incidence generally parallels geographic prevalence of chronic hepatitis B virus (HBV) infection. The presence of HBV increases risk of hepatocellular carcinoma by >100-fold among HBV carriers. Incorporation of HBV-DNA into the host's genome may initiate malignant transformation, even in the absence of chronic hepatitis or cirrhosis.

Other disorders that cause hepatocellular carcinoma include cirrhosis due to chronic hepatitis C virus (HCV) infection, hemochromatosis, and alcoholic cirrhosis. Patients with cirrhosis due to other conditions are also at increased risk. Environmental carcinogens may play a role; eg, ingestion of food contaminated with fungal aflatoxins is believed to contribute to the high incidence of hepatocellular carcinoma in subtropical regions.

Diagnosis may be based on AFP measurement and an imaging test. In adults, AFP signifies dedifferentiation of hepatocytes, which most often indicates hepatocellular carcinoma; 40 to 65% of patients with the cancer have high AFP levels (>400 ?g/L). High levels are otherwise rare, except in teratocarcinoma of the testis, a much less common tumor. Lower values are less specific and can occur with hepatocellular regeneration (eg, in hepatitis). Other blood tests, such as AFP-L3 (an AFP isoform) and des-gamma-carboxyprothrombin, are being studied as markers to be used for early detection of hepatocellular carcinoma. Depending on local preferences and capabilities, the first imaging test may be contrast-enhanced CT, ultrasonography, or MRI. Hepatic arteriography is occasionally helpful in equivocal cases and can be used to outline the vascular anatomy when ablation or surgery is planned.

Various systems can be used to stage hepatocellular carcinoma; none is universally used. One system is the TNM system, based on the following: T: How many primary tumors, how big they are, and whether the cancer has spread to adjacent organs; N: Whether the cancer has spread to nearby lymph nodes; M: Whether the cancer has metastasized to other organs of the body. Numbers (0 to 4) are added after T, N, and M to indicate increasing severity. Other scoring systems include the Okuda and the Barcelona-Clinic Liver Cancer staging systems. In addition to tumor size, local extension, and metastases, these systems incorporate information about the severity of liver disease.

For example, MYC phosphorylation, activation, and thereby tumorigenic potential are regulated by HMG-CoA reductase (see Cao et al. (2011) Cell Research 71(6)2286-1197, herein specifically incorporated by reference). The inhibition of HMG-CoA reductase was found to block MYC phosphorylation, which can be demonstrated by a phosphorylation sensor in situ in human HCC cells.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body. Metastasis may be reduced, for example, by decreasing the numbers of cancer cells in an organ or body part which is not directly connected to the organ of the original cancerous tumor by at least about 10%, at least about 20%, at least about 30%, at least about 50%, at least about 75%, at least about 90%, at least about 95%, at least about 99%, or more.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of hepatocarcinoma, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) inhibiting the disease, i.e., arresting its development; and (b) relieving the disease, i.e., causing regression of the disease.

In particular, treating cancer comprises reducing metastasis and invasiveness of the cancer. For example, the spread of a cancer to sites other then the primary site may be reduced. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a cancer therapeutic drug, anti-cytokine antibodies, etc.

means administration of the agents at such time that the agents each will have a therapeutic effect. Such concomitant administration may involve concurrent (i.e. at the same time), prior, or subsequent administration of the agents with respect to the administration of each other. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

### Compositions and Methods

In some embodiments, the antibody conjugated to the nanoparticle is an F(ab) fragment. In some embodiments the F(ab) fragment is covalently bound to the nanoparticle. In some embodiments the F(ab) fragment is covalently bound to PEG in a nanoparticle comprising PLGA-PEG. In some embodiments the F(ab) fragment is covalently bound through a free thiol group to a maleimide linker joined to PEG in a nanoparticle comprising PLGA-PEG. The F(ab) fragment may be synthesized as such, or may be a digest on the original, intact antibody.

In some embodiments the antibody conjugate is prepared by conjugated F(ab) fragments comprising a free thiol with MAL-PEG-PLGA monomers. The PEG may be of a molecular weight from about 1k to about 20k, e.g. 1k, 2k, 5k, 10, 15k, 20k. The PLGA may be of a molecular weight from about 1k to about 20k, e.g. 1k, 2k, 5k, 10, 15k, 20k. In some embodiments the monomer is MAL-PEG(5k)-PLGA(5k). In some embodiments the antibody fragment is combined with the monomers at a ratio of from about 10:1, 5:1, 2:1, 1:1; 1:2; 1:5; 1:10; 1:20 F(ab) to monomer ratio. The F(ab) conjugated monomer forms nanoparticles by an oil and water emulsion method. The resulting nanoparticles may have an average diameter of from about 100-500 nM, from about 200-300 nM, and may be around 240-270 nM. The potential of the antibody conjugate nanoparticle may be from about −20 to about −30 mV.

The nanoparticles are formulated for therapy in a pharmaceutically acceptable excipient, e.g. in a unit dosage formulation. “Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

Methods are provided for treating or reducing primary or metastatic cancer, particularly metastatic cancer, e.g. adenocarcinomas, colorectal carcinomas; squamous cell carcinomas; basal cell carcinomas; ovarian cancer, pancreatic cancer, breast cancer; etc., and specifically including hepatocellular carcinoma, in a regimen comprising contacting the targeted cells with nanoparticles comprising antibodies specific for checkpoint proteins, e.g. PD-L1. In some embodiments the nanoparticles are F(ab) conjugated antibodies to PLGA nanoparticles with PEG coating.

Methods comprise administering to a subject in need of treatment a therapeutically effective amount or an effective dose of the combined agents of the invention, including without limitation combinations of the agents with a chemotherapeutic drug, radiation therapy, anti-tumor antibody, checkpoint inhibitor, CART cell, etc.

Effective doses of the agent, or combined agents of the present invention for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. Dosage may be commensurate with approved dosing regimens of anti-PD-L1 antibodies as disclosed above. An exemplary treatment regime entails administration once every two weeks, three weeks, 4 weeks, or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor growth, tumor metastasis or tumor invasion of cancers including carcinomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Compositions for the treatment of cancer can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the compositions described herein and instructions for use. The kit can further contain a least one additional reagent, e.g. a chemotherapeutic drug, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially ablate targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples will be described to illustrate parts of the invention. It is also understood that the terminology used herein is for the purposes of describing particular embodiments.

### EXAMPLES

In this work, we investigated whether a checkpoint inhibitor loaded onto a NP could improve the therapeutic efficacy of a checkpoint inhibitor. Specifically, we developed a PEG-PLGA delivery system loaded with the F(ab) fragment of PD-L1 mAb to overcome delivery challenges currently facing PD-L1. We hypothesized that the nanoparticle-antibody conjugates will exhibit improved therapeutic efficacy by achieving high tumor-specific delivery by employing the pharmacokinetics of the PEG-PLGA NPs, extending circulation time of the antibodies by increasing their geometry and removing the Fc portion, and therefore minimize off-target distribution and toxicity. Our results show that PD-L1 NPs improve the therapeutic efficacy of PD-L1 by itself in a MC38 colorectal tumor model. This study establishes checkpoint inhibitor-conjugated NPs are able to overcome mAb delivery challenges seen in the clinic, thus establishing a platform technology able to be extended to multiple mAb types and disease targets.

### Methods

Synthesis and Characterization of PD-L1 F(ab) fragments. PD-L1 (clone 10F.9G2, BioXCell) or CTLA-4 (clone UC10-4F10-11, BioXCell) antibodies were fragmented using established protocols. Briefly, the whole IgG was digested with pepsin (ThermoFisher) according to manufacturer's instruction with minor modifications (16 hour incubation). The F(ab)2 fragment was collected by purifying the digest using a 50 kDa MWCO centrifuge filter (Vivaspin 500), and then further digested into F(ab) fragments with free sulfhydryl groups after incubation with TCEP HCl (20 mM) for 90 minutes. The F(ab) fragments were purified using 10K MWCO centrifuge filters. MALDI-TOF analysis confirmed the successful fragmentation of the antibodies.

PD-L1-PEG-PLGA Conjugation and Formation of Nanoparticles. F(ab) fragments were coupled to MAL-PEG(5k)-PLGA(5k) monomers (Nanosoft Polymers) prepared at 10 mg/mL in 0.1M NaPO₄, 0.15M NaCl, 10 mM EDTA solution by incubating at a 1:10 F(ab) to PEG-PLGA ratio. The coupling was run at 4° C. overnight before purification using 10K MWCO centrifuge filters. To form nanoparticles, the PD-L1-PEG-PLGA monomers were solvent-replaced with DMSO, and then added dropwise into ultra-pure water at a 1:10 v/v ratio. The solution was then homogenized for 5 minutes to form PD-L1-PEG-PLGA NPs. The NPs were purified and concentrated using 10K MWCO centrifuge filters.

Characterization of PD-L1-PEG-PLGA Nanoparticles. The particle size of PD-L1-PEG-PLGA NPs was measured using dynamic light scattering (DLS) on a 90Plus particle size analyzer (Brookhaven Instruments, Holtsville, N.Y.). The particles were diluted in DI water at 1:10 v/v with. After vigorous pipetting to break up aggregates, the NPs were sterile-filtered through syringe filters with a pore size of 200 nm. Raw distribution data was plotted in Graphpad Prism software and fit using a Gaussian curve, with the mean being taken as the particle size for that replicate. The average of three separate replicates was taken to find the mean particle size±standard error of the mean (SEM). We also determined the zeta potential of the particles using a 90Plus Zeta Potential Analyzer (Brookhaven Instruments, Holtsville, N.Y.). Particle formulations were dissolved in DI water at 1:10 v/v. The average of three separate replicates was taken to find the mean zeta potential±SEM.

Cell Culture. MC38 cells (generously donated by the laboratory of Dr. Ronald Levy) were cultured in T-75 flasks using MEM supplemented with 10% FBS, 1% antibiotic-antimycotic, 1% sodium pyruvate, 1% non-essential amino acids, and 1 mL gentamicin. The cells were maintained at 37° C., 5% CO2, and 95% relative humidity and medium was changed every 48 hours. Cells were passaged at 80-90% confluency using a 0.25% trypsin/0.20% EDTA solution. For development of subcutaneous tumors, 1×10⁶ MC38 cells were prepared 1:1 media:Matrigel and injected as a final volume of 150 uL.

Animal Work. Animals were housed in a pathogen-free environment at Stanford University and all procedures were performed in accordance with Stanford's Administrative Panel on Laboratory Animal Care (APLAC) protocols. For biodistribution experiments, nod scid gamma (NSG) mice (4-6 weeks) were injected with 500 ug of PD-L1-PEG-PLGA nanoparticles fluorescently-labeled with a Cy5 dye and imaged using an IVIS Spectrum bioluminescence imager (BLI, Perkin Elmer).

Biodistribution. To measure the distribution of PD-L1-PEG-PLGA nanoparticles, NSG mice were administered either 500 ug of PD-L1, an equivalent amount of PD-L1-loaded PEG-PLGA nanoparticles, or other controls (F(ab)₂, F(ab), and empty PEG-PLGA NPs) intravenously to measure the geographical distribution of the treatments over time. Fluorescence was measured by BLI at time 0, 4, 8, and 24 hours, and organs were excised to measure fluorescence after 24 hours.

Antibody Administration. Mice were enrolled into one of three treatment groups two days following tumor detection. Control IgG and αPD-L1 (clone 10 F.9G2, BioXCell) antibodies were given i.p. (100 μg/mouse) every other day. αCTLA-4 antibody (clone UC10-4 F10-11, BioXCell) was given i.p. (100 μg/mouse) every 3 days.

Tumor Development and Therapy. For therapy experiments, C57BL/6 mice were injected with 1×10⁶ MC38 cells in 1:1 media:Matrigel and observed every other day for palpable tumors. Animals were monitored every other day and palpable tumors were measured using calipers every 2 days thereafter. Once tumors were detected, animals were randomly enrolled into study groups and treatment was begun after 2 days. 100 ug of IgG, anti-PD-L1, or PD-L1-PEG-PLGA NP was injected IP on Day 2 after detection and then every 3 days for total of 3 injections (Days 2, 5, and 8). Tumor growth was monitored for 15 days using a caliper and volume was calculated by V=½(length)*(width)².

Statistical Analysis. Results (mean±SEM) were analyzed for statistical significance by Student's t-test and one-way ANOVA using Graphpad Prism (Graphpad Software, Inc.). Significance is denoted by *for p<0.05, **for p<0.01, and ***for p<0.001.

### Results & Discussion

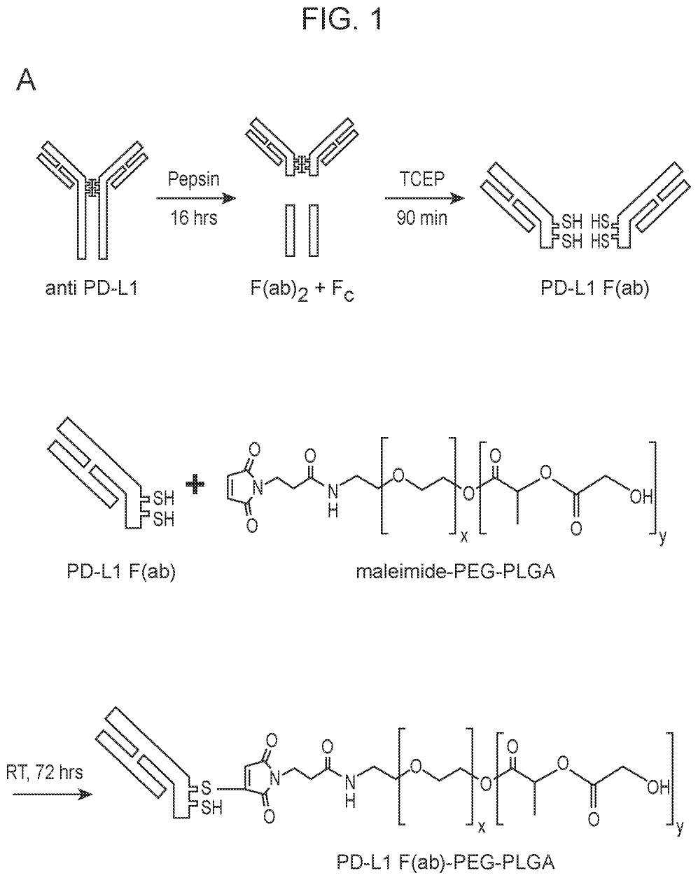

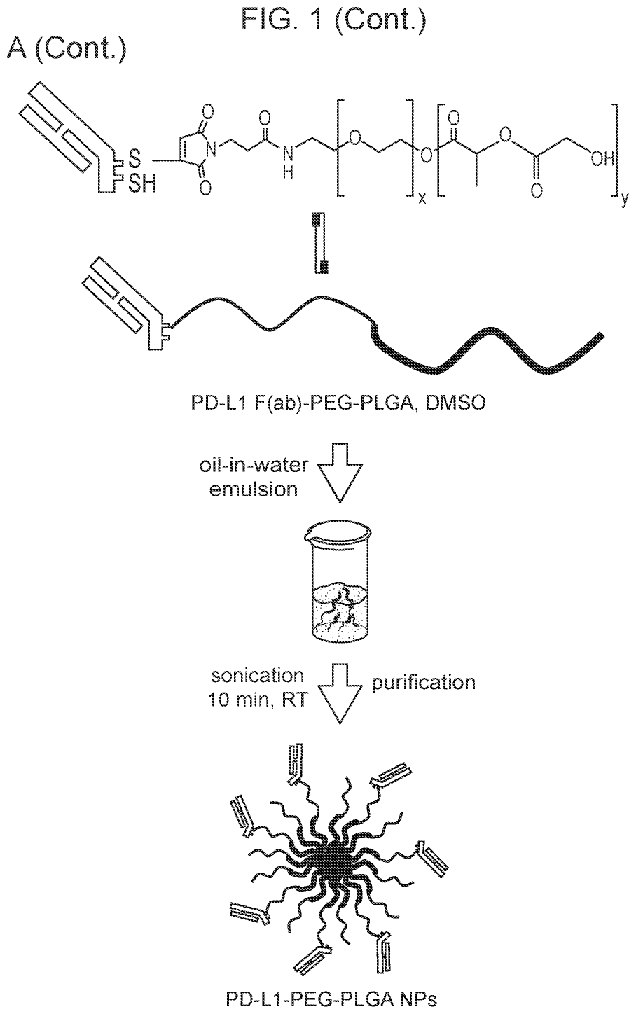

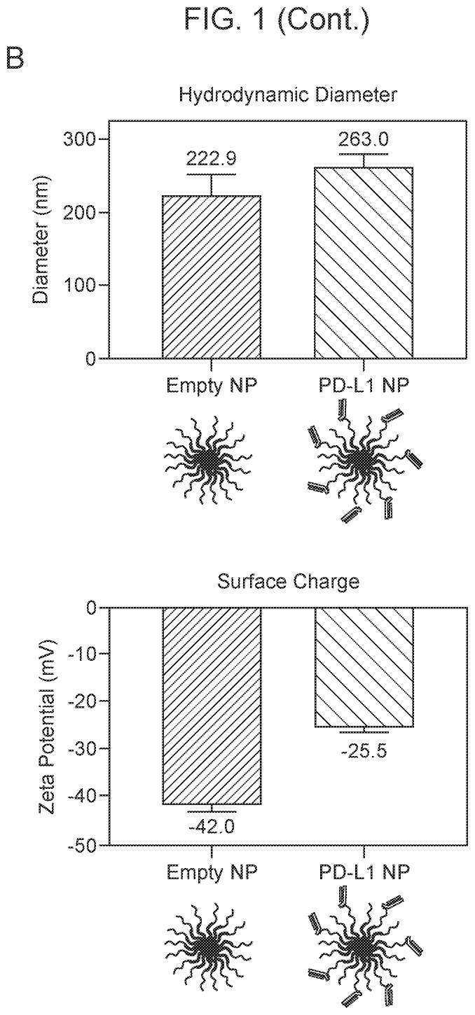

Synthesis and characterization of PD-L1-PEG-PLGA nanoparticles. PEG-PLGA nanoparticles are FDA-approved NP carriers that exhibit high water solubility, biocompatibility, controllable synthesis, and size/weight properties that extend circulation time and allow penetration into tumor tissue. We loaded F(ab) fragments of PD-L1 antibodies to ensure the Fc portion did not induce off-target recognition by the Fc receptor on immune compartments and thereby cause off-target immune-mediated toxicity (FIG. 1A). We successfully fragmented whole PD-L1 antibodies as confirmed by the molecular weights measured by MALDI-TOF. To the F(ab) fragments, we utilized a maleimide linker functionalized to the end of PEG-PLGA monomers to conjugate to the free thiol groups exposed on the F(ab) after reduction. We then formed nanoparticles using a standard oil-in-water emulsion procedure (FIG. 1A) and confirmed the geometry of the NPs using DLS and Zeta Potential measurements (FIG. 1B). It is evident that after attachment to the NPs, the geometry of the antibodies was increased above the limit for renal excretion, ensuring that rapid clearance would be minimized after intravenous injection.

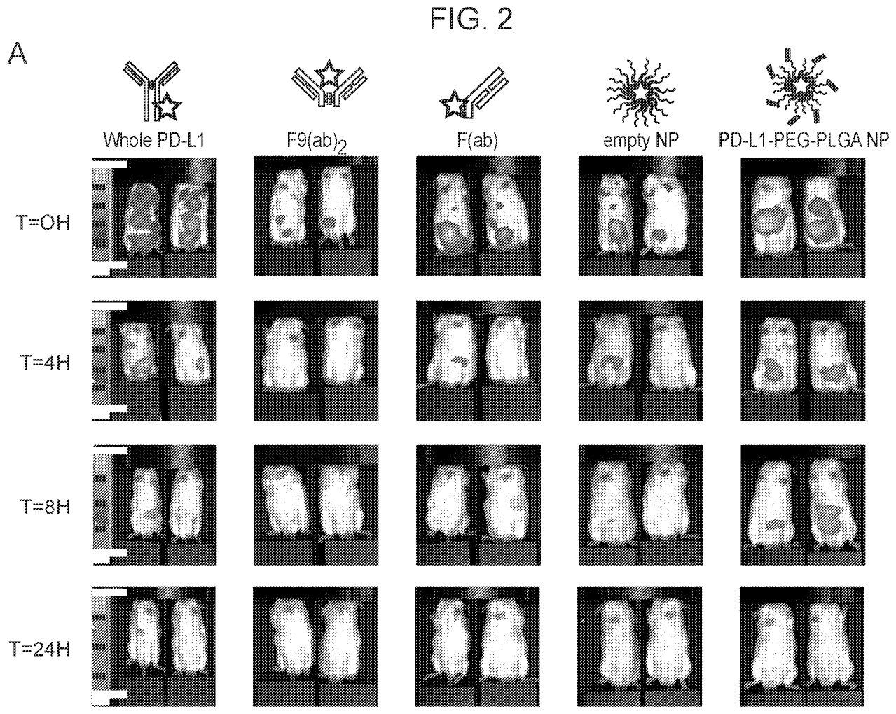

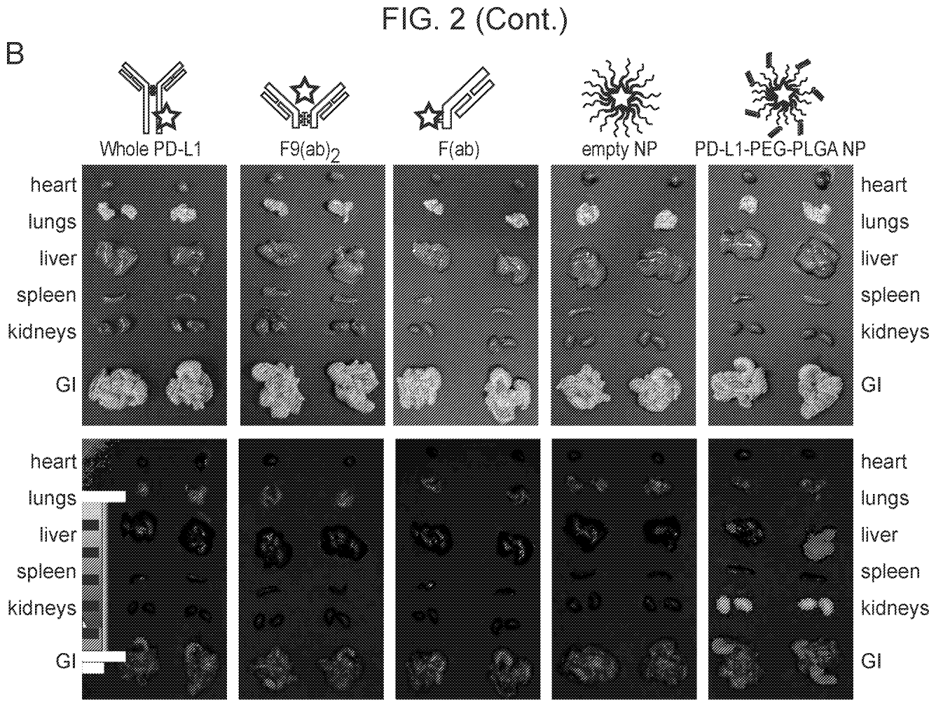

PD-L1-PEG-PLGA nanoparticles extend circulation of antibodies in NSG mice. We measured the distribution of intravenously-injected NPs to test whether loading onto a NP would alter the kinetics and distribution profile of the attached antibodies. We used a Cy5 dye to label whole PD-L1, PD-L1 F(ab)₂, F(ab), empty PEG-PLGA NPs, and PD-L1-PEG-PLGA NPs and achieved equivalent fluorescent labeling (Table 1). The fluorescently-labeled library was then injected intravenously at equivalent Cy5 concentration into NSG mice and monitored via BLI at time=0, 4, 8, and 24 hours. While the majority of fluorescent signal disappeared for the four control groups, a strong signal in the intraperitoneal cavity was maintained for the PD-L1-PEG-PLGA NPs group (FIG. 2A). This was confirmed after excising vital organs and measuring the fluorescence—the liver, kidneys, and GI all exhibit fluorescent intensity in the PD-L1-PEG-PLGA group versus all other control groups (FIG. 2B).

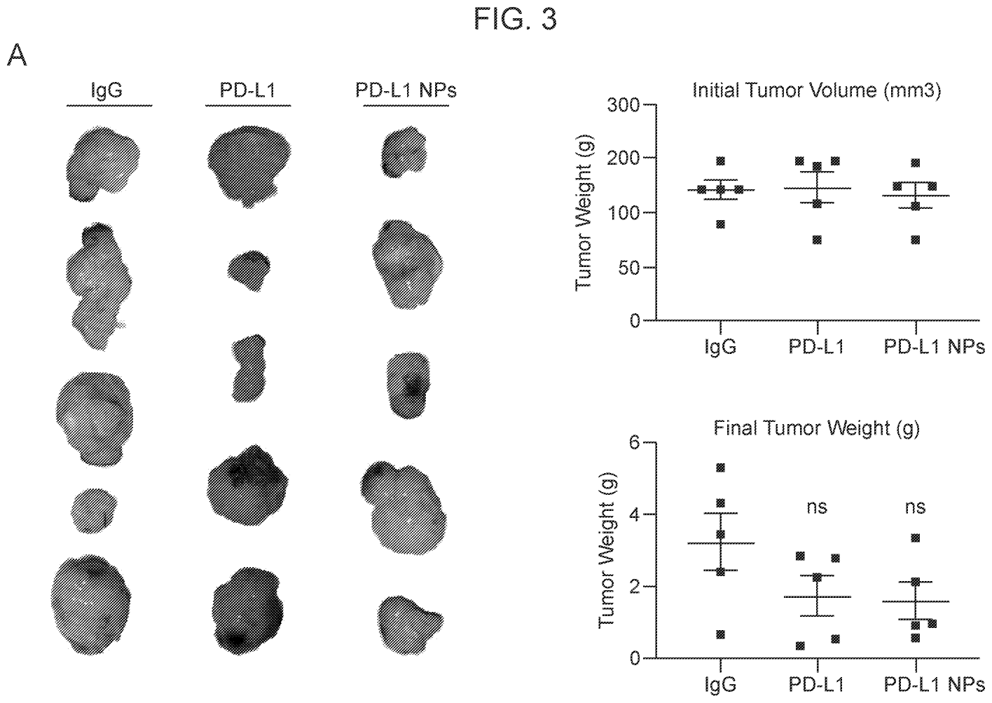

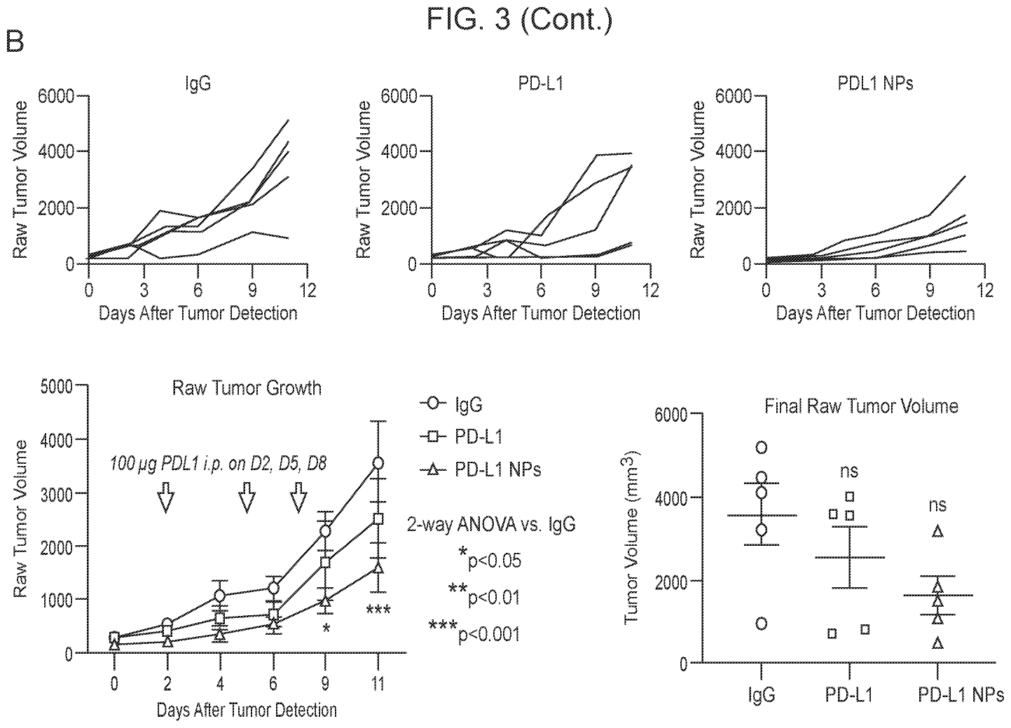

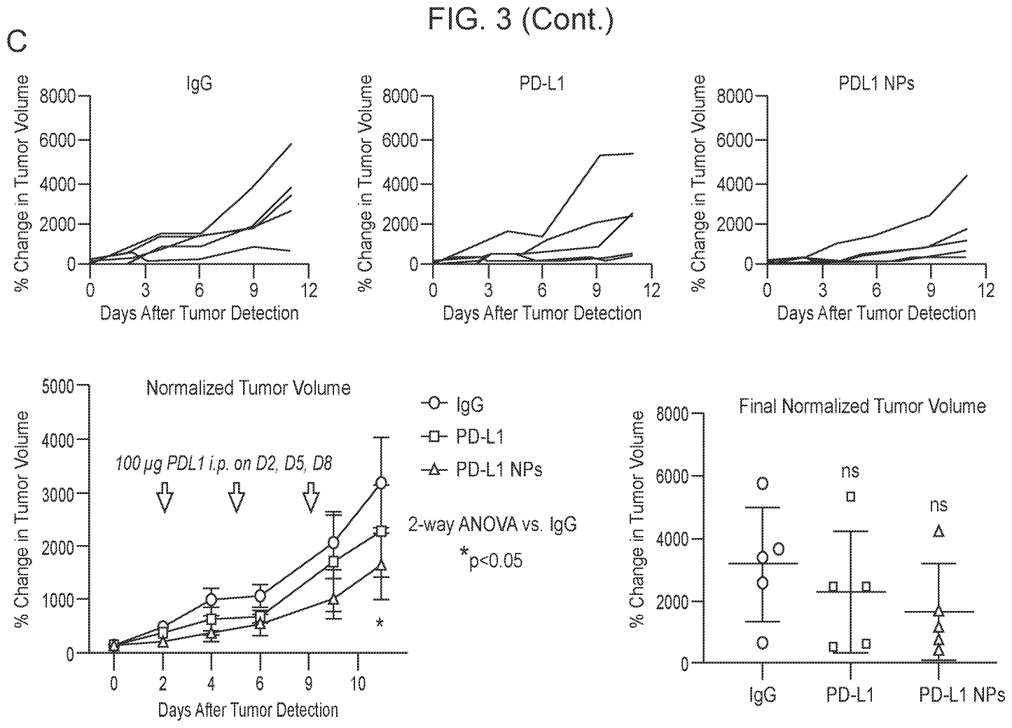

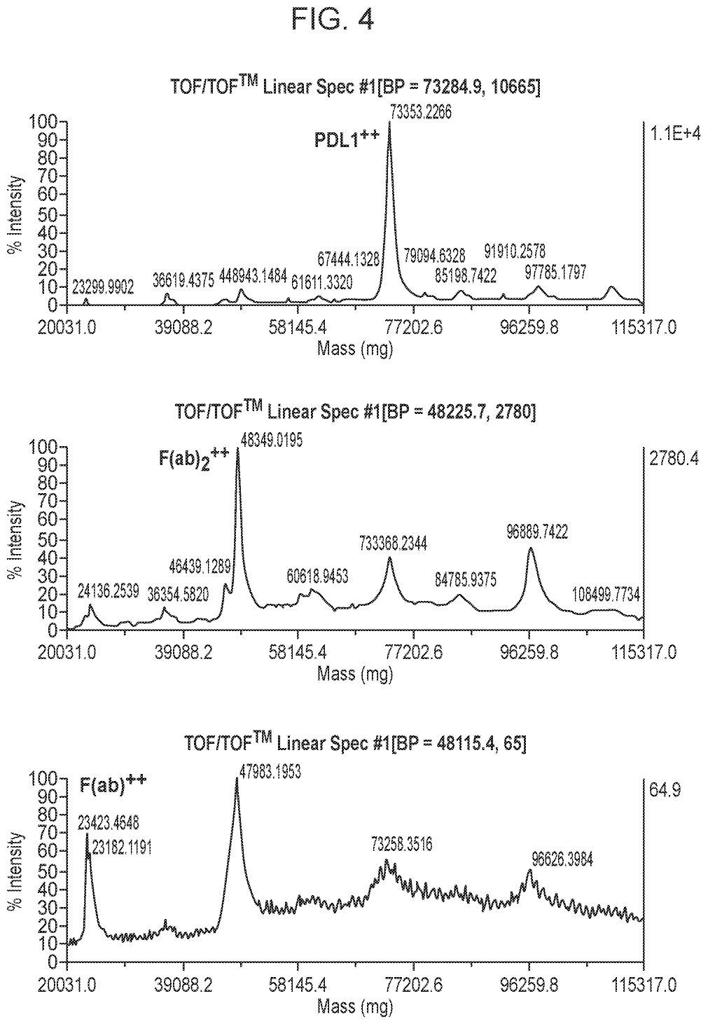

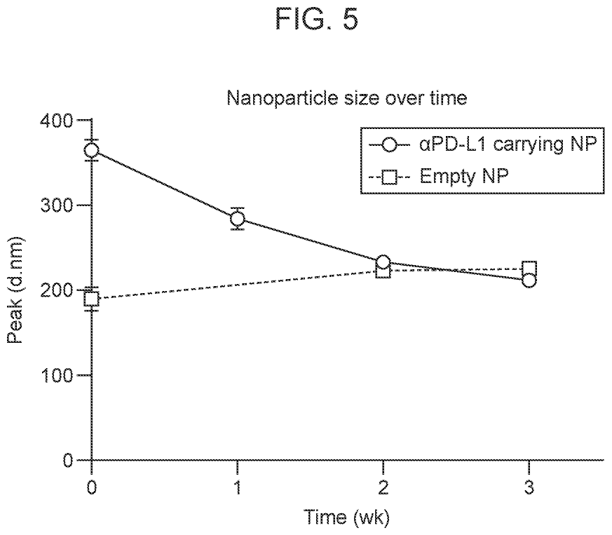

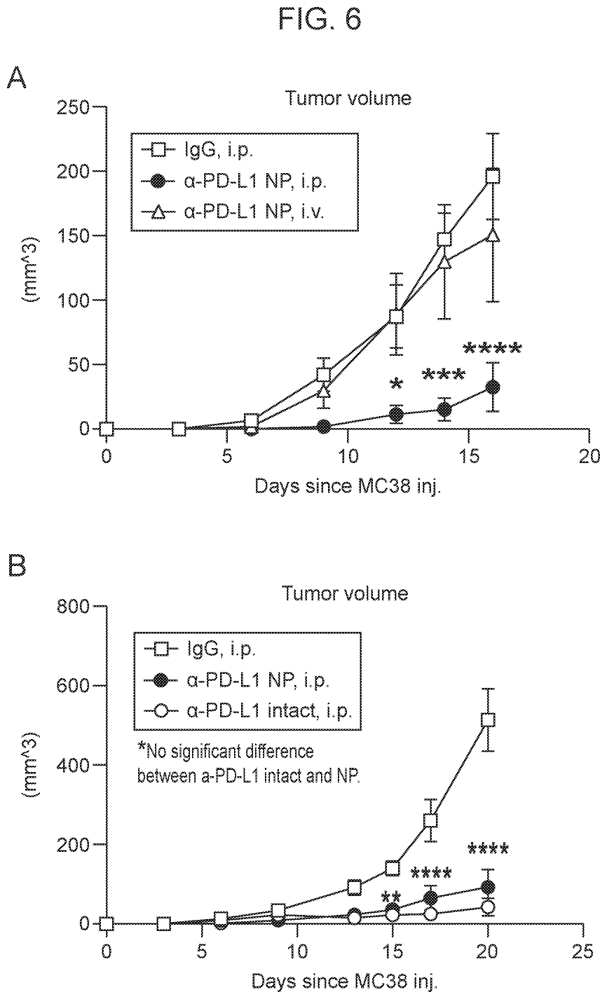

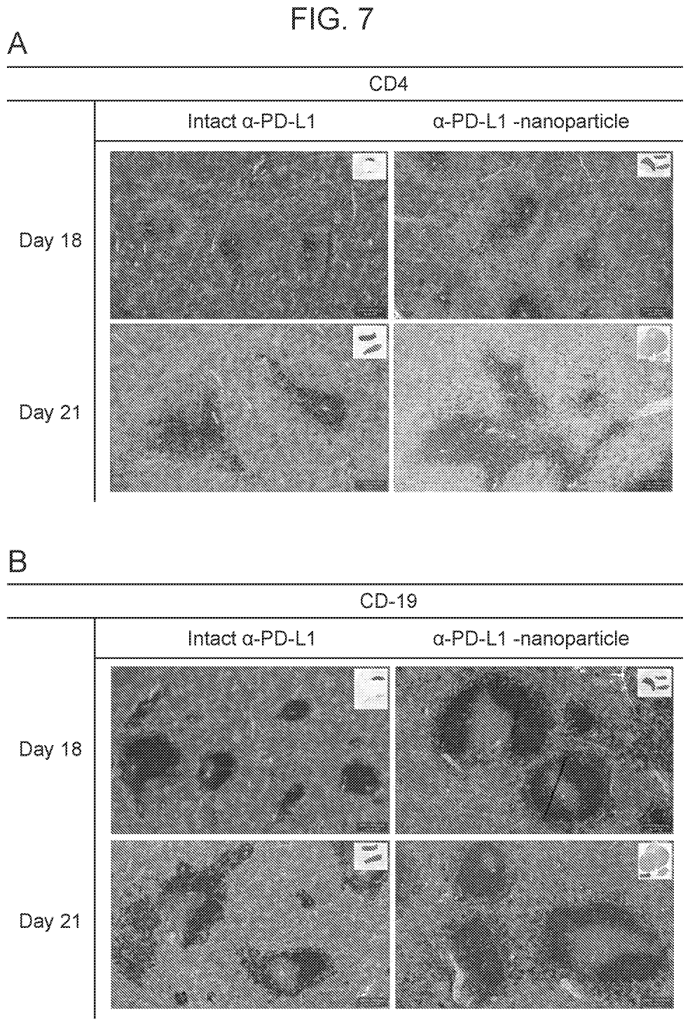

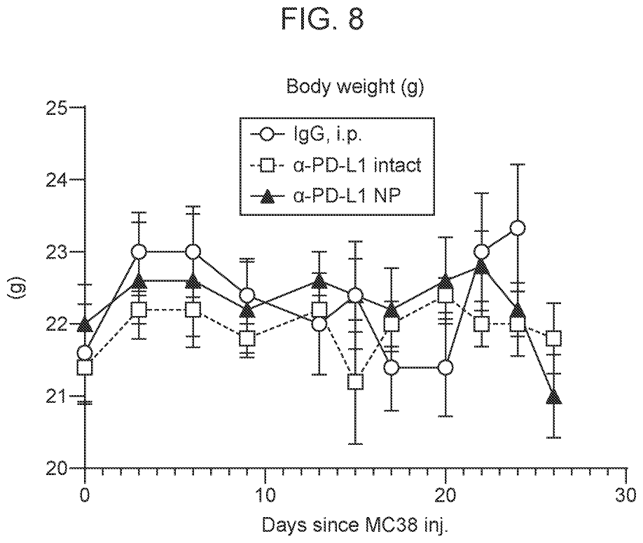

PD-L1-PEG-PLGA nanoparticles improve therapeutic efficacy of PD-L1 in MC38 tumor model. Based on established results in MC38 of PD-L1 monotherapy, we compared the therapeutic efficacy of PD-L1-PEG-PLGA NPs to whole PD-L1 mAbs. Once tumors reached 50-150 mm³ in size after detection, we began treatment with 100 μg PD-L1 administered once every 3 days for 3 total injections. Tumors were monitored for 15 days in total. At the end of the study, tumor volumes did not visibly appear different and tumor weights were not statistically different from IgG controls (FIG. 3A). However, when plotting raw tumor volume growth, there was a significant delay in tumor growth observed in the PD-L1-PEG-PLGA treatment group versus IgG or PD-L1 by itself by Days 9 and 11 (FIG. 3B). After normalizing tumor volume to their volumes at Day 0 (FIG. 3C), significant delay in tumor growth was still observed by Day 11 in the PD-L1-PEG-PLGA NP-treated group.

Although previous reports indicate that PD-L1 monotherapy is effective against MC38 tumors, many reports present variable data. Our findings support these results in that many of the animals respond to monotherapy, while others do not, creating a bimodal response trend and leading the lack of significance from IgG controls. We observed also that NP-treated mice do respond somewhat to treatment compared to IgG controls, but larger numbers of animals and a longer duration of study will help elucidate how strong the therapeutic efficacy is.

This study demonstrates the feasibility and efficacy of attaching PD-L1 antibody fragments onto PEG-PLGA nanoparticles to improve the delivery of mAbs to solid tumor tissue. Our results indicate that PD-L1-PEG-PLGA are synthetically feasible and exhibit amenable physicochemical properties to enable extended circulation time and targeted delivery to typical solid tumor organs like the liver, kidneys, or GI tract. Initial therapy studies indicate that the attachment of PD-L1 F(ab) to PEG-PLGA is able to improve the therapeutic response in an MC38 colorectal tumor model, indicating that the F(ab) is alone able to exhibit immunotherapy and the loading onto PEG-PLGA NPs promotes its efficacy. Taken together, these results suggest that PEG-PLGA NP-mediated delivery of monoclonal antibodies may be a viable means to overcome clinical challenges facing immunotherapy for the treatment of solid tumors.

### Example 2

The size of PEG-PLGA nanoparticle (empty NP) and antibody carrying nanoparticle (α-PD-L1 carrying NP) were measured over 3 weeks with Zetasizer Nano ZS90. Significant decrease in size was exhibited in the antibody carrying nanoparticle. The size of PEG-PLGA nanoparticle (empty NP) and antibody carrying nanoparticle (α-PD-L1 carrying NP) were comparable by week 3. The data highlights the stability of PEG-PLGA over time and the improvements that can be made to make the antibody carrying nanoparticles more stable in long time studies.

The route of therapeutic substance introduction may also play a critical role in tumor suppression. The antibody conjugated nanoparticles were delivered through tail injection (IV) giving direct access to the circulatory system; or by intra-peritoneal injection (IP) which will have to be absorbed through lymphatic tissue before it can enter the circulatory system. It was found that tumor bearing mice that received the antibody carrying nanoparticles through IP had significantly reduced tumor growth compared to tumor bearing mice that received the antibody carrying nanoparticles through IV.

The nanoparticles that travel through host lymphatic system may be able to interact with and educate more immune cells before going to the tumor site. PD-L1 is expressed by a variety of cells including cancer cells, tumor associated macrophages, MSDCs, dendritic cells, T cells and B cells. There have been reports of PD-L1 on dendritic cells attenuating T cell activation and regulating response to immune checkpoint blockade. PD-L1 blockade can either work by enhancing naïve T cell priming in draining lymph nodes, or by reactivating dysfunctional T cells in tumor tissues. Peng et al found that antitumor effects mainly depend on newly activated T cells in the lymph nodes in early stage tumors. However, as the tumors progress, sufficient T cells stay inside tumor tissues but they become more dysfunctional. Then, the antitumor effects depend more on the reactivation of T cells inside tumors. Hartley et al found that treatment of mouse and human macrophages with PD-L1 antibodies increased spontaneous macrophage proliferation, survival, and activation supported by increased in pro-inflammatory costimulatory molecule expression and cytokine production by macrophages. RNAseq analysis revealed macrophage treatment with PD-L1 antibodies upregulated multiple macrophage inflammatory pathways including mTOR pathway activity.

In vivo treatment with PD-L1 antibody resulted in increased tumor infiltration with activated macrophages. Similarly, our antibody carrying nanoparticles that are introduced intra-peritoneally may function initially through activating T cells in the lymphatic tissues early in the tumoregenesis and then by reactivating T cells inside the tumors at later stage. The size modification by attaching the immune checkpoint inhibitor to PEG-PLGA allows the nanoparticles to stay in the host system longer.

Antibody carrying nanoparticles confer extended immune response in tumor bearing mice, relative to intact PD-L1. CD4+ and B cells population persist weeks after antibody treatment only in antibody carrying nanoparticle treated mice group.

Based on the increased immunological changes we observed in spleens collected from tumor bearing mice that received antibody carrying nanoparticle, we were concerned that antibody carrying nanoparticles may confer unintentional side effects to host. However, we did not find any significant changes in mice weight and behavior between the groups that received the antibody carrying nanoparticle and the intact antibody treatment.

Peng, Q., Qiu, X., Zhang, Z. et al. PD-L1 on dendritic cells attenuates T cell activation and regulates response to immune checkpoint blockade. Nat Commun 11, 4835 (2020).

Hartley, G. P., Chow, L., Ammons D. T. et al. Programmed Cell Death Ligand 1 (PD-L1) Signaling Regulates Macrophage Proliferation and Activation. Cancer Immunol Res 6 (10), 1260-1273 (2018).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

What is claimed is:

1. A PLGA-PEG nanoparticle covalently linked to a antigen-binding fragment lacking an Fc region of an antibody specific for a checkpoint inhibitor.

2. The PLGA-PEG nanoparticle of claim 1, wherein the antibody specifically binds to human PD-L1 protein.

3. The PLGA-PEG nanoparticle of any of claims 1-2, wherein the antibody is selected from Atezolizumab; Avelumab; and Durvalumab.

4. The PLGA-PEG nanoparticle of claim 1, wherein the antibody specifically binds to human PD-1 protein.

5. The PLGA-PEG nanoparticle of claim 4, wherein the antibody is selected from Pembrolizumab; Nivolumab; spartalizumab; Cemiplimab; Camrelizumab; and Toripalimab.

6. The PLGA-PEG nanoparticle of any of claims 1-5, wherein the antibody fragment is an F(ab) fragment, conjugated to PEG.

7. The PLGA-PEG nanoparticle of any of claims 1-6, wherein the nanoparticle is comprised of the antibody fragment conjugated to PEG(5k)-PLGA(5k) monomers.

8. The PLGA-PEG nanoparticle of any of claims 1-7, wherein the nanoparticle has an average diameter of from about 100-500 nM.

9. A pharmaceutical formulation comprising an effective dose of a nanoparticle according to any of claims 1-8, and a pharmaceutically acceptable excipient.

10. The formulation of claim 9, wherein the effective dose is from about 500 to about 2000 mg antibody.

11. A method of treating an individual for cancer, the method comprising:
administering an effective dose of a nanoparticle of formulation thereof according to any of claims 1-10 to the individual.

12. The method of claim 11, wherein the administration is by intravenous or intraperitoneal delivery.

13. The method of claim 11, wherein administration is intraperitoneal.

14. The method of any of claims 11-13, wherein the cancer is a carcinoma.

15. The method of claim 14, wherein the cancer is hepatocellular carcinoma.

16. The method of any of claims 11-15, wherein the treatment results in reduced tumor growth relative to treatment with an unconjugated antibody.

17. The method of any of claims 11-15, wherein the treatment provides for extended time of effectiveness relative to treatment with an unconjugated antibody.
